(19) United States
(12) Patent Application Publication
(10) Pub. No.: US 2006/0210480 A1
(43) Pub. Date: Sep. 21, 2006

(54) Treatment of reduction of dysphagia

(76) Inventor: Shaheen Hamdy, Wilmslow (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/085,705

(22) Filed: Mar. 21, 2005

(51) Int. Cl. A61N 1/00; A61K 49/00; A61K 31/4745

(52) U.S. Cl. 424/10.1; 514/305; 607/2

(57) ABSTRACT

The present invention provides methods of treating or reducing dysphagia in patients. In one aspect of the invention, such method comprises administering to a patient in need of such treatment or reduction an amount of an agent sufficient to produce a therapeutically effective aversive taste response. In other aspects, such methods comprise inducing in a patient in need of such treatment or reduction a therapeutically effective aversive taste response as well as administering to the patient a further swallowing stimulus.

### BACKGROUND OF THE INVENTION

The present invention relates to methods for the prevention of dysphagia and/or the treatment of dysphagia.

Dysphagia is the condition whereby a patient has difficulty in swallowing, or is unable to swallow. Dysphagia may be caused, for example, by stroke, neurodegenerative diseases, brain tumours or in some case by other co-morbidity such as respiratory disorders.

Swallowing is a rigidly ordered sequence of events that results in the propulsion of food from the mouth through the pharynx and oesophagus to the stomach. At the same time, respiration is inhibited and food is prevented from entering into the trachea. Swallowing can be initiated voluntarily, but thereafter it is almost entirely under reflex control. The swallowing reflex is typically initiated by sensory impulses from tactile receptors (particularly those located near the opening of the pharynx) being transmitted to certain areas in the medulla. The central integrating areas for swallowing lie in the medulla and lower pons; they are collectively called the swallowing centre. Motor impulses travel from the swallowing centre to the musculature of the pharynx and upper oesophagus via various cranial nerves. This lower swallowing centre in the brainstem is under regulatory control by higher centres in the cerebral cortex. These higher swallowing centres or regions control the voluntary initiation and modulation of the swallow.

Swallowing occurs in three stages. In the oral or voluntary phase, food is moved towards the back of the mouth by the tongue, and forced into the pharynx, where it stimulates the tactile receptors that initiate the swallowing reflex.

In the pharyngeal stage of swallowing, food passes through the pharynx by constriction of the walls of the pharynx, backward bending of the epiglottis, and an upward and forward movement of the larynx and trachea. During the pharyngeal stage, respiration is reflexively inhibited.

In the oesophageal stage of swallowing, food moves down the oesophagus and into the stomach, assisted by one or more peristaltic waves.

Although the main function of swallowing is the propulsion of food from the mouth into the stomach, swallowing also serves as a protective reflex for the upper respiratory tract, preventing unwanted particles from entering the tract. For instance, dysphagia after a stroke can be a devastating problem, as it carries a six fold increased risk of aspiration pneumonia.

In the past, a number of separate treatments have been utilised to assist patients in regaining adequate swallowing reflexes.

For instance, some patients have undergone thermal stimulation treatment. In thermal stimulation treatment, the tonsillar fossa is stimulated by a cooled mirror or probe, and the patient closes his mouth and attempts to swallow.

Alternatively, International Patent Application No. PCT/US96/17015 describes a method for treating dysphagia with electrical stimulation. The application describes a non-invasive method for treating dysphagia and artificially promoting swallowing by electrical stimulus. A plurality of electrodes are selectively placed in electrical contact with skin over the neck, around the pharyngeal region of the patient, and a series of electrical pulses applied to the electrodes. The electrical pulses selectively stimulate muscle located proximate to the electrodes, to beneficially alter swallowing function. The patent application describes how patients treated by electrical stimulation recovered the ability to swallow at a faster rate than those patients treated by thermal stimulation.

It is an aim of embodiments of the present invention to provide methods suitable for the treatment or reduction of dysphagia. Such methods of the invention will preferably constitute therapeutic techniques for facilitating the process of recovery of patients from dysphagia.

### SUMMARY OF THE INVENTION

A method of treating or reducing dysphagia according to one embodiment of the present invention comprises administering to a patient in need of such treatment or reduction an amount of an agent sufficient to produce a therapeutically effective aversive taste response. Other embodiments also provide improved methods of treating or reducing dysphagia in patents.

Related objects and advantages of the present invention will be apparent from the following drawings and detailed description.

### BRIEF DESCRIPTION OF THE DRAWINGS

FIG. 1 shows the results of Example 1 illustrating the ability of quinine to increase excitability associated with the swallowing response;

FIG. 2 shows the results of Example 2, confirming the ability of orally administered quinine to increase the amplitude of pharyngeal motor evoked potential (MEP) indicative of the swallowing response;

FIG. 3 shows the results of Example 3, illustrating that quinine administered directly to the stomach is able to increase the amplitude of pharyngeal MEP indicative of the swallowing response;

Table 1 provides a numeric summary of the results of Example 2; and

Table 2 provides a numeric summary of the results of Example 3.

### DESCRIPTION OF PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations, modifications, and further applications of the principles of the invention being contemplated as would normally occur to one skilled in the art to which the invention relates.

Accordingly, in a first aspect of the invention there is provided a method of treating or reducing dysphagia, the method comprising administering to a patient in need of such treatment or reduction an amount of an agent sufficient to produce a therapeutically effective aversive taste response.

In a second aspect the invention further provides a method of treating or reducing dysphagia, the method comprising administering to a patient in need of such treatment or reduction a therapeutically effective amount of an agent having an aversive taste.

In a third aspect, the invention provides a method of treating or reducing dysphagia, the method comprising inducing in a patient in need of such treatment or reduction a therapeutically effective aversive taste response.

It is known that most mammals will eat beyond their homeostatic needs if a particular food is tasty, that is to say a food having a pleasant taste. This illustrates that certain foods, particularly those having tastes generally associated with nutritious properties (such as the provision of caloric energy or dietary salt) are intrinsically rewarding.

The invention is based on the inventors surprising finding that administration to patients of substances having an aversive taste is able to induce a swallowing response, and to thereby treat or reduce dysphagia. It may be expected that pleasant or positive taste stimuli, such as sweet tasting substances, may stimulate swallowing. The inventors have found that, in contrast to expectations, aversive tastes are notably more effective in inducing a swallowing response. Although they do not wish to be bound by any hypothesis the inventors suggest that the effectiveness of aversive taste stimuli may arise as a result of their “unpleasantness” which is able to induce greater sensory stimulation. In addition, the inventors believe that aversive taste stimuli may excite cortical pathways associated with swallowing responses.

A patient receiving treatment by means of the methods of the invention may be a person or an animal. In the context of the present invention a swallowing stimulus should be taken to be any stimulus that initiates, facilitates or improves the retention of the swallowing reflex.

It will be appreciated that the treatment of dysphagia in accordance with the present invention, should be taken to encompass any therapeutic decrease in the level of dysphagia up to and including full recovery from dysphagia. Similarly, the reduction of dysphagia should be taken to encompass any therapeutic decrease in the level of dysphagia exhibited by a patient.

Treatment or recovery from dysphagia in patients undergoing therapy with methods in accordance with the invention may be assessed with reference to any decrease in the level of dysphagia that may typically occur in control or untreated patients to whom therapy has not been administered. Alternatively a decrease in dysphagia exhibited by patients undergoing therapy with methods in accordance with the invention may be assessed by a clinician, most suitably the clinician in charge of care of the patient in question, with reference to the clinician's own experience of the decrease in dysphagia that may be exhibited by untreated patients, and/or with published accounts of the decrease in dysphagia that may be found in untreated patients.

Preferably treatment or reduction in accordance with the methods of the invention may give rise to a decrease in dysphagia at least 5%, 10%, 20% or 30% greater than a decrease occurring in a control or untreated individual. More preferably treatment or reduction in accordance with the methods of the invention may give rise to a decrease in dysphagia at least 40%, 50% or 60% greater than a decrease that may occur in a control or untreated individual. It is even more preferred that treatment or reduction in accordance with the methods of the invention may give rise to a decrease in dysphagia at least 70%, 80%, or 90% greater than a decrease occurring in a control or untreated individual, and most preferably treatment or reduction in accordance with the methods of the invention may give rise to a decrease in dysphagia at least 100% greater than any decrease that may occur in control or untreated individuals.

In the present context a therapeutically effective amount of an agent for use in the methods of the present invention will be an amount sufficient to bring about an alleviation or reduction in dysphagia suffered by a patient. For instance, a therapeutically effective amount of an agent suitable for use in accordance with the methods of the invention may be an amount sufficient to induce swallowing by the patient.

Swallowing presence or absence of swallowing response in a patient undergoing therapy using methods in accordance with the invention may be readily assessed physically, in that the physical swallowing action may be easily observed. Alternatively or additionally, the presence or absence of a swallowing response may be assessed using suitable monitoring means. The development of dysphagia post stroke, and it's subsequent recovery, has been associated with changes in the excitability of the swallowing motor cortex, as for instance described within the article “Recovery of swallowing after dysphagic stroke relates to functional reorganization in the intact motor cortex. Gastroenterology 115: 1104-1112, 1998 by Shaheen Hamdy et al. Accordingly suitable monitoring means may include means able to assess cortical and/or muscular activity associated with swallowing, for example by means such as electromyogram (EMG) readings.

Such monitoring means may be advantageous in that they are able to detect “partial” swallowing responses (i.e. incomplete swallowing reflexes that are not of sufficient magnitude to give rise to a full swallowing response) and thus may be able to detect increases in the swallowing reflex brought about by methods of the invention even when such increases are not sufficient to induce a swallowing response. It will be appreciated that even such incomplete swallowing responses may be of therapeutic value as they may be augmented using other swallowing stimuli.

A therapeutically effective aversive taste response will be an aversive taste response sufficient to bring about an alleviation or reduction in dysphagia, such as by inducing swallowing by the patient. A taste response may be defined as the generation of any signal (including intracellular and neuronal signals) indicative of the stimulation of taste receptors.

For the purposes of the present invention aversive tastes should be taken to encompass both bitter tastes and sour tastes. The perception of bitter or sour tastes is well conserved across humans. Typically aversive tastes are associated with foodstuffs that are not beneficial to an animal eating them. Bitter tastes are generally associated with foods that are toxic, while sour tastes are typically associated with foods that have spoiled or decayed.

The gustatory system has been shown to allow the detection of noxious or toxic chemicals (which typically have aversive bitter or sour tastes) at submicromolar levels, whereas compounds providing caloric energy (which typically have pleasant sweet tastes) can only be detected at concentrations in the submolar range. In the light of this knowledge, it will be appreciated that the use of aversive stimuli in methods according to the invention is advantageous, given the relatively greater sensitivity of the gustatory system to such stimuli.

Agents suitable for use in accordance with the methods of the invention that have aversive bitter tastes include quinine; quinine hydrochloride; extracts of wormwood; cycloheximide; phenylthiocarbamide (PTC); and denatonium benzoate (sold commercially under the brand name Bitrex). The alkaloid quinine, and its pharmaceutically acceptable salt quinine hydrochloride are preferred agents suitable for use in accordance with the invention. Except for where the context requires otherwise references to the use of quinine in the present specification should also be taken to encompass the use of quinine hydrochloride.

It has recently been suggested that bitter taste is sensed through the activity of the T2R family of bitter taste receptors. This family of taste receptors comprises members that are believed to represent the products of at least 30 related genes. The T2R receptor family are thought to signal via the phospholipase C (PLC) signal transduction pathway, and evidence suggests that PLC components such as PLCβ2 are necessary for the perception of bitter taste. Thus one suitable definition of bitter agents that may be used in the methods of the invention is agents that are capable of binding to a receptor of the T2R family and eliciting a signalling response. The skilled artisan will readily be able to investigate the ability of a test compound of interest to elicit such a signal using suitable in vitro or in vivo models well known in the prior art.

Examples of agents suitable for use in the methods of the invention that have aversive sour tastes are generally acidic in nature, and include such compounds as lactic acid, malic acid, acetic acid, and naturally occurring fruit acids, such as citrus acid.

The perception of sour taste is linked to an increase in the concentration of protons in the vicinity of a sour-sensing taste bud. This increase in proton concentration, provided by acids in foods, leads to the generation of a sour taste. Thus agents suitable for use in the methods of the invention having sour taste may readily be identified by their ability to increase proton concentration in a manner able to elicit a sour taste response in a test subject.

An agent suitable for use in the methods of the invention may preferably be introduced into the upper digestive tract, for the purposes of the present invention considered to comprise the portion of the digestive tract extending inclusively from the mouth to small intestine. In the event that it is wished to induce an aversive taste response in a patient, or to administer an agent capable of eliciting such an aversive taste response it may be preferred to introduce the requisite agent into the mouth of the patient, more preferably onto the tongue of the patient, and most preferably onto the rear of the tongue. It will however by appreciated that agents introduced downstream of the tongue or mouth may also achieve an aversive taste response, for example by reflux or other flow into the patient's mouth.

The inventors have surprisingly found that therapeutic agents such as quinine are able to stimulate swallowing even when administered to the digestive tract “downstream” of the taste sensing cells located in the mouth. By way of example, the inventors have found that administration of quinine directly into the stomach, for example by infusion, is a particularly effective manner by which to stimulate the swallowing response. Although they do not wish to be bound by any hypothesis, the inventors believe it is possible that quinine may activate taste receptors in the small bowel. Such stimulation may take place through an enteroendocrine process involving hormones such as cholecystokinin (CCK), which plays a role in feeding and satiety. Accordingly the administration of agents such as quinine into the stomach represents a preferred feature of methods in accordance with the invention.

In a fourth aspect of the invention there is provided a method of treating or reducing dysphagia, the method comprising administering to a patient in need of such treatment or reduction a therapeutically effective amount of quinine.

In a fifth aspect of the invention there is provided a method of treating or reducing dysphagia, the method comprising administering to a patient in need of such treatment or reduction a therapeutically effective amount of an alkaloid.

Quinine may be naturally isolated, such as from the bark of Cinchona succirubra, or it may be artificially synthesised according to well-known protocols. It is known that quinine is only one of 31 related alkaloids that may be isolated from Cinchona succirubra, and it will be recognised that alkaloids other than quinine selected from this group may be used effectively in accordance with the methods of the invention (and in particular with the fifth aspect of the invention).

It is preferred that agents used in the methods of the invention be administered at a concentration sufficient to induce an aversive taste response in the patient to which they are administered. In the event that such agents are administered in the form of a solution suitable concentrations for use in the methods of the invention range from between 0.01 mM and 1 M depending on the nature of the agent administered. The inventors have found that solutions of between 0.02 mM and 0.1 mM of the bitter tasting aversive agent quinine constitute preferred concentrations of solutions suitable for use in the methods of the invention. Most preferably quinine may be administered at a concentration of 0.05 mM for use in methods according to the invention.

It will thus be appreciated that agents suitable for use in accordance with the methods of the invention may be formulated using a liquid pharmaceutical vehicle. Suitable pharmaceutical compositions may be in the form of a solution or suspension comprising the selected agent. Liquid vehicles may be used in preparing solutions, suspensions, emulsions, syrups, elixirs and pressurized compositions comprising agents suitable for use in the methods of the invention. Such agents may be dissolved or suspended in a pharmaceutically acceptable liquid vehicle such as water, an organic solvent, a mixture of both, or pharmaceutically acceptable oils or fats. The liquid vehicle can contain other suitable pharmaceutical additives such as solubilizers, emulsifiers, buffers, preservatives, suspending agents, thickening agents, colours, viscosity regulators, stabilizers or osmo-regulators.

Suitable examples of liquid vehicles by which agents for use in the methods of the invention may be administered to a patient include water (which may further contain thickening agents such as cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil).

Liquid formulations comprising agents suitable for use in the methods of the invention may be administered to a patient in need of treatment or reduction of dysphagia sporadically (for instance as drops of the formulation), or, if appropriate, administered more constantly (for instance as a stream of the formulation).

As an alternative to the use of solutions of agents suitable for use in accordance with the methods of the invention it may be preferred to use solid compositions from which the agent may be liberated, for example by the action of the patient's saliva or gastric juices. Suitable solid compositions capable of yielding therapeutically effective amount of agents suitable for use in the methods of the invention will be readily apparent to those of skill in the art, but may, by means of non-limiting example, include tablets, pastilles, pellets, or capsules. Alternatively the agents to be used in methods of the invention may be administered in the form of coatings on digestible or indigestible substrates suitable for administration to patients undergoing treatment or reduction of dysphagia.

Accordingly, a pharmaceutically acceptable vehicle for use with an agent suitable for effecting the methods of the invention might be a solid, and a suitable composition may be in the form of a powder or tablet. As set out above, such formulations may preferably be soluble in the saliva or gastric juices of a patient to whom they are administered.

Suitable solid vehicles may further include one or more substances that may also act as lubricants, solubilizers, suspending agents, fillers, glidants, compression aids, binders or tablet-disintegrating agents. Solid vehicles may also comprise an encapsulating material. In powders, the vehicle is a finely divided solid that is in admixture with the finely divided agent to be used in the method of the invention. In tablets, the agent is mixed with a vehicle having the necessary compression properties in suitable proportions and compacted in the shape and size desired. Preferred powders and tablets may contain up to 99% of an agent suitable for use in the methods of the invention. Suitable solid vehicles include, for example, calcium phosphate, magnesium stearate, talc, starch, gelatin, cellulose, polyvinylpyrrolidine, low melting waxes and ion exchange resins.

Liquid or solid formulations comprising agents suitable for use in accordance with the invention may be administered to a patient by means of a spray or aerosol. Suitable means by which such agents may be administered include oral inhalers, pump sprays, and pressurized compositions. Liquid vehicle for pressurized compositions may suitably be a halogenated hydrocarbon or other pharmaceutically acceptable propellant.

Preferably the agents to be used in methods of the invention may be administered in a form such that the patient receives repeated dosages of the agent. The inventors have found that repeated administration of agents at periods ranging from between 1 and 120 seconds may be useful in effecting the methods of the invention. Preferably repeated administration may be effected at periods from between 2 and 30 seconds, and most preferably repeated administration of the agents may be effected every 15 seconds.

In cases where the methods of the invention are effected by administration of suitable agents in liquid formulations such repetitious administration may readily be effected by repeated administration of distinct boli of the liquid formulation. In cases where the methods of the invention are effected by administration of suitable agents in the form of solid formulations repetitious administration may be achieved by the use of alternated layers (such as in tablets or coated substrates) in which the requisite agent is alternately provided and absent, thereby producing an alternating effect as subsequent layers are dissolved.

It is preferred that a swallowing response be induced repeatedly in a patient by use of the methods of the invention. For example, it will normally be preferred to induce multiple swallowing responses in a single session of treatment. Such sessions of treatment may involve the induction of between 10 and 100 swallowing responses, preferably between 20 and 70 swallowing responses, more preferably between 30 and 50 swallowing responses and most preferably approximately 40 swallowing responses.

That said, treatment or reduction of dysphagia in accordance with the methods of the invention appears to alter the brain activity of a patient so treated in a way that is likely to prove beneficial to the recovery of the patient. It will thus be appreciated that treatment in accordance with the methods of the invention may be effective in helping to bring about a therapeutic improvement in swallowing function even in the absence of swallowing (i.e. treatment may promote the recovery or strengthening of the swallowing response even when the treatment itself does not induce swallowing).

Although single sessions of treatment may be effective in the treatment or reduction of dysphagia, it will generally be preferred that multiple sessions of treatment as described above be combined to produce a course of treatment. Such courses of treatment may be continued until a patient's dysphagia has been completely treated, or until sufficient beneficial reduction of dysphagia has been achieved (which may, for example, be assessed by a physician responsible for the treatment of a patient).

Methods in accordance with the present invention may be used as the sole therapeutic modality in the treatment or reduction of dysphagia (i.e. treatment or reduction of dysphagia brought about only through the action of methods of the invention). Alternatively the methods of the invention may be used as adjuncts to known or novel therapeutic regimes for the treatment or reduction of dysphagia (i.e. treatment or reduction of dysphagia brought about by the combined activity of methods of the invention combined with other therapeutic regimes or agents).

The inventors have found that, while the methods of the invention are capable of treating or reducing dysphagia when used as the sole therapeutic regime, the combination of methods of the invention with other swallowing stimuli is particularly advantageous. Accordingly in preferred embodiments of the invention the methods of the invention may additionally comprise administering to the patient a further swallowing stimulus.

It may be preferred that the further swallowing stimulus is administered to the patient at the same time as administration of the agent. In such embodiments the patient may thus receive a swallowing stimulus induced by the method of the invention at the same time as a further swallowing stimulus (such as an electrical, magnetic or temperature swallowing stimulus).

A suitable further swallowing stimulus may be administered to the patient prior to administration of the agent. Thus the patient may receive the further swallowing stimulus (such as an electrical, magnetic or temperature swallowing stimulus) before the swallowing stimulus induced by the method of the invention.

Alternatively, a suitable further swallowing stimulus may be administered to the patient after administration of the agent. Accordingly the patient may receive a swallowing stimulus induced by the method of the invention, followed by the further swallowing stimulus (such as an electrical, magnetic or temperature swallowing stimulus).

It will be appreciated that more than one further swallowing stimulus may be administered to a patient in combination with the methods of the invention. Accordingly a patient may receive a swallowing stimulus provided by the method of the invention in combination with two, three or more further swallowing stimuli. The considerations outlined above regarding timing of administration will also apply to the administration of multiple further swallowing stimuli. The timing of multiple further swallowing stimuli may be coordinated such that the patient receives all further stimuli at the same time (which may or may not be at the same time as the swallowing stimulus induced by the method of the invention), or it may be arranged such that the patient receives the further swallowing stimuli independently of one another (and which may also be independently of the swallowing stimulus induced by the method of the invention).

Suitable swallowing stimuli capable of use in combination with the methods of the invention include intraluminal electrical pharyngeal neuromuscular stimulation and/or transcranial magnetic stimulation (TMS). Suitable TMS may comprise repetitive TMS and may be applied directly to the cortex. In addition, transcranial direct current stimulation, via electrodes applied to the scalp, may also be used advantageously in combination with the methods of the invention. Further stimuli suitable for combination with the methods of the invention include visual stimuli (such as visual representations of food or feeding) and olfactory stimuli (which may be in the form of strong food odours).

Accordingly, preferred embodiments of the methods of the invention may further comprise administering to the patient electronic stimulation capable of inducing or augmenting a swallowing response. A preferred example of suitable electronic stimulation that may be used in combination with the methods of the invention may be achieved by the use of electrodes capable of providing intra-luminal pharyngeal stimulation in a patient to be treated. Such electrodes are typically connected to an electrical signal generator capable of generating a series of electrical pulses capable of acting to provide a swallowing stimulus. The frequency and duration of such pulses, and power and/or current, suitable for generating a swallowing stimulus will be well known to those of skill in the art.

Preferably suitable electrical pulses may be generated at a predetermined frequency in the range of 0.05 to 5 Hertz. Suitable pulses may have duration in the region of 200 milliseconds. Electrical pulses suitable for use in combination with the methods of the invention may have a current of between 1 and 50 milliamps and may be delivered at a voltage of 280 millivolts.

The duration of time over which a series of electrical pulses may be applied to a patient requiring treatment or reduction of dysphagia may be selected with reference to the extent of dysphagia exhibited by the patient. Typically a therapeutic effect may be achieved utilising a train of electrical pulses administered for a period of between 10 and 30 minutes.

Other preferred embodiments of the methods of the invention may further comprise administering to the patient magnetic stimulation capable of inducing or augmenting a swallowing response. Transcranial magnetic stimulation provides a suitable method by which areas of the cortex associated with swallowing may be stimulated.

Preferably magnetic swallowing stimuli for use in combination with the methods of the invention may be provided by the use of an electromagnet able to provide TMS. Examples of suitable magnetic stimulators capable of generating a swallowing stimulus include the Magstim200 and Magstim Super Rapid stimulator, manufactured by MAGSTIM Company Ltd, Witland, Wales. A suitable swallowing response may be generated using magnetic stimulation achieving an output in the region of 2.2 Tesla, preferably using a frequency of 5 Hz.

Still other preferred embodiments of the methods of the invention may further comprise administering to the patient temperature stimulation capable of inducing or augmenting a swallowing response. Temperature stimulation encompasses the use of either raised or lowered temperature as a stimulus. Thus suitable temperature stimulation may comprise the application of a hot or cold stimulus effective to induce or augment a swallowing response.

The methods of the invention may be used to treat or reduce dysphagia at any time after the onset of the condition. However, the methods of the invention may preferably be utilised within the first month after onset of dysphagia, more preferably within the first week after onset, and most preferably within 24 hours after the onset of dysphagia.

The methods described thus far have been described with reference to the treatment or reduction of dysphagia, that is to say in the context of achieving a therapeutic effect with respect to an existing condition of dysphagia. However, it will be readily appreciated that the methods herein described are suitable for use in the prevention of dysphagia. The application of the methods of the invention in this preventative manner is particularly useful in the case of patients with a predisposition to dysphagia, or those at elevated risk of developing dysphagia. Examples of patients who may benefit from the preventive use of the methods of the invention include patients that have suffered from strokes neurodegenerative disease or brain tumours, or patients undergoing oro-pharyngeal anaesthesia. The preventive use of the methods of the invention may also be advantageous for patients suffering from other co-morbidity, such as respiratory disorders, associated with increased risk of dysphagia, and patients with cricopharyngeal dysfunction such as those with gastro-oesophageal reflux, and motility disorders of the pharynx and oesophagus.

The preventive use of methods in accordance with the invention may preferably be employed as soon as the elevated risk of developing dysphagia is recognised. In the case of patients where the increased risk of dysphagia arises as a result of a medical condition, such as stroke, the preventive treatment using methods of the invention may be commenced as soon as possible after the onset of the medical condition, or as soon as possible after the medical condition is recognised or diagnosed. In the case of patients at increased risk of dysphagia as a result of oro-pharyngeal anaesthesia treatment, reduction or prevention of dysphagia using the methods of the invention may be initiated shortly before, at the same time as, or shortly after administration of the oro-pharyngeal anaesthetic.

It will be appreciated by the skilled artisan that the effects of dysphagia may lead a patient suffering from dysphagia to develop further deleterious conditions. Accordingly it will be recognised that the methods of the invention may be of utility in preventing the onset or development of such diseases caused by dysphagia. Thus in a sixth aspect of the invention there is provided a method of preventing a disease caused by dysphagia by effecting treatment or reduction of dysphagia using a method as described in the first to fifth aspects of the invention. One disease that arises as a particularly frequent and damaging result of dysphagia is aspiration pneumonia, and accordingly in a seventh aspect of the invention there is provided a method of preventing or treating aspiration pneumonia caused by dysphagia, the method comprising treating a patient with a method as described in the first to fifth aspects of the invention.

The invention will now be described further with reference to the accompanying examples and drawings:

### EXAMPLE 1

An assessment was made as to the effects of different taste stimuli on human cortical swallowing pathways. The assessment was made upon 8 healthy adult volunteers (seven of which were male, the minimum age of the volunteers being 29 years).

Each volunteer performed a ten-minute, liquid swallowing task using three (previously titrated) different taste solutions. The three solutions utilised were sterile water (neutral taste), 10% glucose (sweet/pleasant taste), and 0.5 mM quinine hydrochloride (bitter/aversive taste). Solutions were randomised to separate studies at least 24 hours apart.

Transcranial magnetic stimulation was performed over the swallowing motor cortex, before and up to one hour after each swallow task in accordance with established protocols. Briefly, Single pulse (monophasic) transcranial stimulation (sTMS) of the cerebral cortex was achieved using a magnetic stimulator (a Magstim 200, manufactured by MAGSTIM Company Limited, Whitland, Wales) connected to a 70 mm outer diameter figure-of-8 coil (maximal output of 2.2 Tesla (T)), held in an A-P (Anterior-Posterior) direction, at an angle of 45° tangential to the scalp.

Repetitive (biphasic) transcranial magnetic stimulation (rTMS) of the cerebral cortex was achieved using a Magstim Super Rapid stimulator (manufactured by MAGSTIM Company Limited, Whitland, Wales) connected to a 70 mm outer diameter figure-of-8 coil (maximal output of 1.8 Tesla (T)), held in an identical orientation to sTMS.

Cortico-pharyngeal motor responses were recorded from a swallowed intra-luminal catheter as a measure of cortical excitability. Responses were then compared using repeated measures ANOVA (Analysis of Variance).

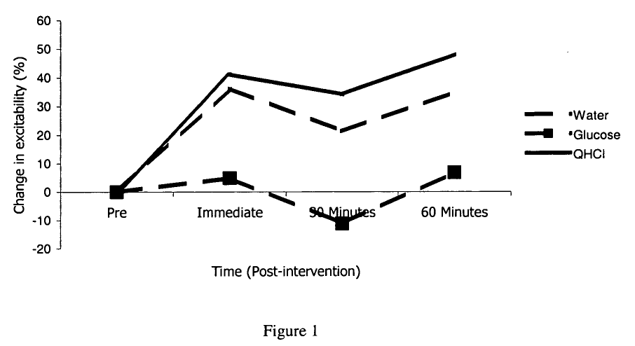

FIG. 1 shows the percentage change in pharyngeal motor evoked potential (MEP) amplitude from the base line MEP measurement at different time intervals prior to and after the swallowing task had been completed. It can be seen that cortico-pharyngeal responses for neutral water were increased, but only in the period immediately after swallowing (percentage change from baseline equals 36±15%, p<0.04, where p is the level of significance and taken to be relevant at <0.05). At 30 minutes, whilst still raised, the water effect was no longer significant. Following the glucose swallowing task, no substantial changes in response are observed.

However, following the swallowing task using the aversive tasting alkaloid quinine, patients exhibited response that were increased compared to those exhibited following the water task, both immediately and throughout the 60 minutes post-intervention period (maximum percentage change from base line equals 48±11%, p<0.01).

### EXAMPLE 2

Confirmation of the suitability of averse tasting substances such as the alkaloid quinine to stimulate the swallowing response and thereby aid treatment or reduction of dysphagia was obtained from a second study measuring swallowing reflexes of volunteers receiving neutral, pleasant or aversive taste stimuli.

Volunteers receive a ten minute infusion of neutral (water), pleasant (glucose) or aversive (quinine) taste stimuli administered such that they stimulated the taste receptors of the tongue.

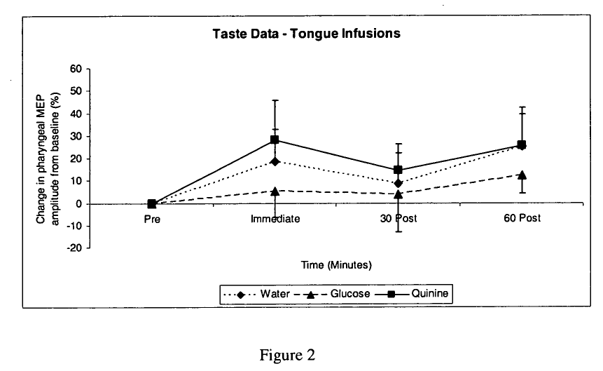

The results (shown in FIG. 2 and Table 1) clearly illustrate that, taken over a 60 minute period calculated from administration of the test taste stimulus, the aversive tasting alkaloid quinine induced a greater increase in the amplitude of MEP (illustrative of a greater increase in stimulation of the swallowing response) than did neutral tasting water or pleasant tasting glucose.

These results confirm the utility of aversive tasting agents such as the alkaloid quinine in the promotion of swallowing and recovery or treatment of dysphagia.

### EXAMPLE 3

The ability of substances such as the averse tasting alkaloid quinine to stimulate the swallowing response when administered into the portion of the upper digestive tract below the mouth was in a third study measuring swallowing reflexes of volunteers receiving neutral, pleasant or aversive taste stimuli.

Volunteers receive a ten minute infusion of neutral (water), pleasant (glucose) or aversive (quinine) taste stimuli administered directly into the volunteer's stomach.

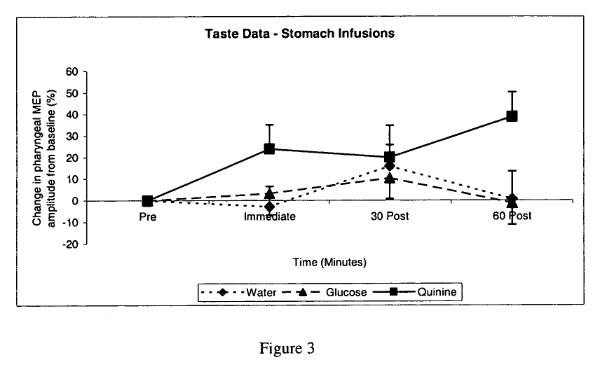

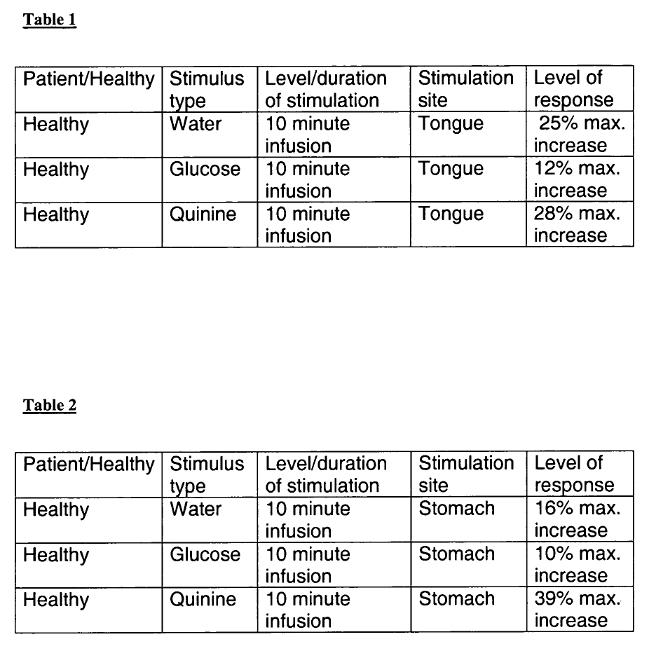

The results (shown in FIG. 3 and Table 2) clearly illustrate that the pro-swallowing effects of the aversive tasting alkaloid quinine are surprisingly increased (as compared to the effects of neutral tasting water or pleasant tasting glucose) when the test compounds are administered directly to the stomach.

As shown in FIG. 3, the increase in MEP induced by quinine is markedly greater than that attained using either neutral water or pleasant tasting glucose. Table 2 illustrates that quinine administered directly to the stomach is able to induce a 39% increase in the swallowing response (measured by MEP) as opposed to a 16% increase induced by water and a 10% increase induced by glucose.

These results indicate that alkaloids such as aversive tasting quinine are surprising able to stimulate the swallowing response (illustrating that such agents may be useful in the treatment or reduction of dysphagia) even when administered directly into the stomach.

The results here reported indicate that cortical swallowing pathways are modulated in a differential manner by pleasant and aversive tasting stimuli. In comparison to neutral stimuli, aversive tastes are more enhancive to the cortical swallowing responses, whereas pleasant tastes may suppress these pathways. It can be seen that aversive taste stimuli (such as those induced by the alkaloid quinine) can be utilised as method for rehabilitating swallowing problems after cerebral injury.

The exact mechanism by which the cortico-pharyngeal responses were increased by quinine may also involve additional factors, other than taste. For instance, it may be that quinine provides a chemical stimulation directly to the gut. It may be that the application of quinine is linked to the release of endocrine hormones such as CCK from the upper GI tract. Such release may be linked to an induction of feeding behaviour such as swallowing. Accordingly, it may be preferable to release quinine at other locations within the body other than the mouth, or even to release endocrine hormones (or other stimulators of such hormones) into the body with nutrients such as C12 fatty acids (i.e. fatty acids with chains consisting of 12 carbon atoms) or other fat based solutions.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A method of treating or reducing dysphagia, the method comprising administering to a patient in need of such treatment or reduction an amount of an agent sufficient to produce a therapeutically effective aversive taste response.

2. A method according to claim 1, wherein the agent is an agent having a bitter taste.

3. A method according to claim 1, wherein the agent is selected from the group comprising quinine; quinine hydrochloride; cycloheximide; phenylthiocarbamide; and denatonium benzoate

4. A method according to claim 1, wherein the agent is an agent having a sour taste.

5. A method according to claim 1, wherein the agent is selected from the group comprising citric acid lactic acid, malic acid and acetic acid.

6. A method according to claim 1, further comprising administering to the patient a further swallowing stimulus.

7. A method according to claim 1, further comprising administering to the patient a further swallowing stimulus administered at the same time as administration of the agent.

8. A method according to claim 1, further comprising administering to the patient a further swallowing stimulus administered after the administration of the agent.

9. A method according to claim 1, further comprising administering to the patient a further swallowing stimulus administered prior to administration of the agent.

10. A method according to claim 1, further comprising administering to the patient electronic stimulation capable of inducing a swallowing response.

11. A method according to claim 1, further comprising intra-lumenal pharyngeal stimulation to the patient.

12. A method according to claim 1, further comprising administering to the patient magnetic stimulation capable of inducing a swallowing response.

13. A method according to claim 1, further comprising administering transcranial magnetic stimulation to the patient.

14. A method according to claim 1, further comprising administering to the patient temperature stimulation capable of inducing a swallowing response.

15. A method according to claim 1, further comprising administering a cold swallowing stimulus to the patient.

16. A method of treating or reducing dysphagia, the method comprising administering to a patient in need of such treatment or reduction a therapeutically effective amount of an agent having an aversive taste.

17. A method of treating or reducing dysphagia, the method comprising inducing in a patient in need of such treatment or reduction a therapeutically effective aversive taste response.

18. A method of treating or reducing dysphagia, the method comprising administering to a patient in need of such treatment or reduction a therapeutically effective amount of quinine.

19. A method of treating or reducing dysphagia, the method comprising administering to a patient in need of such treatment or reduction a therapeutically effective amount of an alkaloid.
